(19) United States
(12) Patent Application Publication
(10) Pub. No.: US 2010/0190845 A1
(43) Pub. Date: Jul. 29, 2010

(54) METHOD OF TREATING CANCER USING ATP SYNTHASE INHIBITORS

(75) Inventors: Hsueh-Fen Juan, Taipei City (TW); Hsin-Yi Chang, Taipei City (TW); Tsui-Chin Huang, Taipei City (TW); Chun-Hua Hsu, Taipei City (TW); Wen-Hung Kuo, Taipei City (TW); King-Jen Chang, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/359,549

(22) Filed: Jan. 26, 2009

(51) Int. Cl. A61K 31/366; A61P 35/00

(52) U.S. Cl. 514/456; 514/460

(57) ABSTRACT

The present invention is directed to a method of treating cancer in a subject in need of such a treatment by administering an inhibitor of ATP synthase in a pharmaceutically effective amount, preferably, the inhibitor contains one pyrone ring. The present invention also provides for a pharmaceutical composition comprising an inhibitor of ATP synthase, preferably, the inhibitor is aurovertin B.

### BACKGROUND OF THE INVENTION

1. Field of the Invention

The present invention relates to a method of treating cancer using ATP synthase inhibitors. More particularly, the present invention relates to novel method useful in the treatment of, for example, breast cancer and lung cancer.

2. The Prior Arts

Cancer has become the number one killer disease after the 20th century. According to statistics made by the department of Health in Taiwan in June 2002, malignant cancer has been the first leading cause on top 10 causes of death for the last 20 years since 1982, with a mortality rate of 147.68 per 100,000 populations, and approximately 33,000 deaths in 2001. Searches for effective anti-cancer compounds with mild side effects therefore become imperative.

Breast cancer is the most common malignancy among women in developed regions of the world. In the United States, more than 200,000 women are diagnosed with breast cancer each year and nearly 41,000 patients die of the disease.

Lung cancer remains the most prevalent cancer in the world, accounting for 30% of all cancer-related deaths. The current prognosis for patients with lung cancer is poor. The overall cure rate is estimated as low as 13%. Approximately 180,000 new cases of lung cancer are expected in the United States in 1999. The majority of these patients will die of their disease with 160,000 deaths from lung cancer expected nation-wide in 1999.

ATP synthase is the enzyme catalyzing the synthesis of ATP. ATP synthase consists of two subcomplexes: F₀ and F₁. F₁ consists of five different polypeptide chains with the stoichiometry α₃β₃γδε. The F₀ subcomplex 11 different subunits and forms a hydrophobic unit that spans the inner mitochondrial membrane. For a long time, F₁F₀ ATP synthase expression was believed to be found only in mitochondria where most cellular ATP synthesis takes place. Besides ATP production, recent studies suggest that components of ATP synthase exist on the outer surface of the plasma membrane where they function as receptors for various ligands and are involved in biological processes such as metabolism of lipid formation, regulation of the proliferation and differentiation in endothelial cells and recognition of immune responses of tumor cells.

Although some evidences indicate that ATP synthase is expressed on the extracellular surface of endothelial cells in some cancer tissues, but its function with regard to cancer development is still unclear. So far no investigation has been conducted to explore the possible therapeutic application of inhibitors of ATP synthase in breast cancer treatment and lung cancer treatment.

The invention provides a method of treating cancer using ATP synthase inhibitors. ATP synthase is a recognition molecule in breast cancer cells and lung cancer cells and allows for the use of ATP synthase inhibitor of the invention, for use cancer chemotherapy.

### SUMMARY OF THE INVENTION

The present invention is directed to a method of treating cancer in a subject in need of such a treatment by administering an inhibitor of ATP synthase in a pharmaceutically effective amount, preferably, the inhibitor contains one pyrone ring. The present invention also provides for a pharmaceutical composition comprising an inhibitor of ATP synthase, preferably, the inhibitor is aurovertin B.

Aurovertin B belongs to the aurovertin family which exhibit toxic property and is a metabolite isolated from the fungus Calcarisporium arbuscula. Aurovertin contains an α-pyrone (or 2-pyrone), a six-membered cyclic unsaturated ester. The derivatives of α-pyrone are widely distributed in nature and some of them inhibit ATP synthase by targeting F₁. Known as an ATP synthase inhibitor, aurovertin B acts to prevent the attainment of the tight conformation in the ATPase cycle.

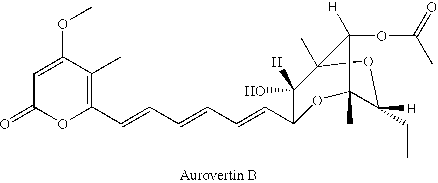

In one aspect, the present invention provides a method for inducing apoptosis in a cancer cell, the method comprising contacting the tumor cell with an inhibitor of ATP synthase activity, wherein the inhibitor is aurovertin B.

In one embodiment, the cancer is breast cancer. In another embodiment, the cancer is lung cancer.

In one embodiment, the cancer is located in a human subject.

In one embodiment, the cancer is resistant to anticancer agent and/or radiation therapy.

In one embodiment, the inhibitor activates the caspases dependent apoptotic pathway and induces cell cycle arrest.

In one embodiment, the inhibitor directly interacts with ATP synthase F₁ subunit.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 Comparison of the 2D and 3D view of ATP synthase β subunit prepared from normal breast cells and breast cancer cells. The expression of ATP synthase β subunit was highly increased in cancer tissues.

FIG. 2 Characterization of ATP synthase expressed on MCF-7 breast cancer cell surface. (A) Confocal microscopy image of the distribution of ATP synthase β subunit on MCF-7 cell surface. MCF-7 were fix and with (lower)/without (upper) permeabilization. Red: ATP synthase β subunit; blue: hoechst 33342. (B) FACS analyzed MCF-7 cells expressing ATP synthase β subunit. Cells in red area represent the ones stained with anti-ATP synthase β subunit Ab followed by FITC-labeled anti-mouse IgG antibodies. Cells in black area denote negative control cells.

FIG. 3 Homology modeling of human ATP synthase and the binding mode of three inhibitors to ATP synthase. (A) Homology modeling of human ATP synthase F1 subunit. The different chains are represented in various colors. This model was used to serve as the target protein for docking simulation to estimate the drug affinity with human ATP synthase. (B) aurovertin B (D) resveratrol (F) piceatannol were docked into the ATP synthase β subunit catalytic site. Hydrophobic (white), negatively-charged (red), and positively-charged (blue) regions are shown. The 3-D pictures were obtained using Discovery Studio 1.2. (C) aurovertin B (E) resveratrol (G) piceatannol molecules are shown to form hydrogen bonds with ATP synthase β subunit.

FIG. 4 Effects of ATP synthase inhibitors on cell viability in MCF-7 breast cancer cells. MTT assay was performed to characterize the specificity of ATP synthase inhibitors on tumor cells. MCF-7 cells were treated with three chemicals, seprarately, for 48 hours. Aurovertin B caused significant cytolysis in MCF-7. On the other hand, cells treated with the other two ATP synthase inhibitors (resveratol and piceatannol) had 80% and higher cell viability rate at the same concentrations and the same time points compared with aurovertin B.

FIG. 5 Effects of aurovertin B on cell viability in breast normal cells (MCF-10A) and cancer cells in vitro. MTT assay was performed to characterize the specificity of aurovertin B on tumor cells (MCF-7, MBAMD231 and T47D). Aurovertin B caused significant cytolysis in breast cancer cell lines. However, normal breast cell line MCF-10A cells treated with aurovertin B had 80% or higher cell viability rate at the same time points, meaning it exerts little toxicity to normal cells.

FIG. 6 Aurovertin B induces cell cycle arrest. (A) Cell cycle analysis of MCF-7 cells treated with 0.1 μM, 1 μM and 5 μM aurovertin B for 48 hours was performed by flow cytometry. (B) The percentage of cells in the G0/G1, S and G2/M phase was calculated. MCF-7 cells cultured in the medium without aurovertin B for 48 hours served as the control. In aurovertin B treated cells, percentage of cells in G0/G1 phase was changed from 63.1% to 66.5%, 78.5%, 81.2% at 0 μM, 0.1 μM, 1 μM, 5 μM concentrations, respectively.

FIG. 7a Aurovertin B induces apoptosis of MCF-7. Cells treated with 0.05 μM, 0.1 μM, 1 μM, 5 μM and 10 μM aurovertin B for 48 hours were double stained with annexin-V-FITC and propidium iodide (PI) and analyzed by flow cytometry. Lower left quadrant shows viable cells; lower right, Annexin-V positive cells (early apoptosis); upper left, cell positive for PI (necrosis); upper right, cell positive for both Annexin-V and PI (late apoptosis). FIG. 7b The percentage of cells in the four quadrants was calculated. MCF-7 cells cultured in the medium without aurovertin B for 48 hours served as the control.

FIG. 8 Characterization of aurovertin B induced cell death in human MCF-7 cells. Upper: phase-contrast microscopy shows cell shrinkage, irregularity in shape, and cellular detachment in aurovertin B-treated cells. Lower: MCF-7 cells stained with 4,6-diamidino-2-phenylindole (DAPI).

FIG. 9 Effects of aurovertin B on cell viability in CL1-1 lung cancer cells. Cell number counting was performed to characterize the specificity of aurovertin B on tumor cells (CL1-1 and CL1-5). Aurovertin B caused significant cytolysis in lung cancer cells, especially in CL1-1 cells. However, normal lung cell line IMR-90 cells treated with aurovertin B had 60% or higher cell viability rate at the same time points, meaning it exerts little toxicity to normal cells.

FIG. 10 Effects of resveratrol on cell viability in CL1-5 lung cancer cells. Cell number counting was performed to characterize the specificity of resveratrol on tumor cells (CL1-1 and CL1-5). Resveratrol caused significant cytolysis in lung cancer cells, especially in CL1-5 cells. However, normal lung cell line IMR-90 cells treated with resveratrol had 60% or lower cell viability rate at the same time points.

FIG. 11 Effects of piceatannol on cell viability in lung cancer cells. Cell number counting was performed to characterize the specificity of piceatannol on tumor cells (CL1-1 and CL1-5). Piceatannol had no cytolysis in lung cancer cells. CL1-1 and CL1-5 cells had 80% or higher cell viability rate. Further, normal lung cell line IMR-90 cells treated with piceatannol had 60% cell viability rate at the same time points.

### DETAILED DESCRIPTION OF THE PREFERRED EMBODIMENT

Definitions:

As used herein, the term “subject,” “individual” or “patient” is used interchangeably herein, which refers to a vertebrate, preferably a mammal, more preferably a human.

By “a pharmaceutically effective” amount of a drug or pharmacologically active agent or pharmaceutical formulation is meant a nontoxic but sufficient amount of the drug, agent or formulation to provide the desired effect.

The term “inhibit growth of cancer (tumor) cells” refers to any action that may decrease the growth of a cancer cell. The inhibition may reduce the growth rate or the size of cancer cells, or inhibit or prevent proliferation, growth of cancer cells. The inhibition may inhibit the colony formation of cancer cells due to the anchorage-independent growth. Preferably, such an inhibition at the cellular level may reduce the size, or deter the growth of a tumor (cancer) in a patient.

The present invention provides for a method of treating breast cancer or lung cancer.

In a preferred embodiment of the present invention, an inhibitor of ATP synthase activity is administered to a subject in need of such a treatment in a pharmaceutically effective amount.

The present invention also provides for a method for inducing apoptosis in a cancer cell comprising contacting an inhibitor of ATP synthase with the cancer cells. Preferably, the inhibitor contains one pyrone ring. More preferably, the cancer cell is a breast cancer cell or lung cancer cell.

Preferably, the cancer is resistant to anticancer agent and/or radiation therapy.

The inhibitors disclosed herein, which retain the binding specificity to ATP synthase β subunit, are also included in the present invention. More preferably, the inhibitor is aurovertin B.

In addition, ATP synthase β subunit was found to differentially express on the surface of some tumor cell lines. Our data show that ATP synthase β subunit is highly expressed on the surface of the breast cancer cells, and the lung cancer cells. However, ATP synthase β subunit is low expressed in the colorectal cancer cells as described in J. M. Cuezva, “The bioenergetic signature of cancer: a marker of tumor progression,” (Cancer Res. 62(22):6674-81(2002)). Moreover, it have been reported that the specific ATP synthase inhibitor aurovertin B didn't affect the release of cytochrome c and apoptosis in Hela cells (Acta Biochimica Polonica 52(2):553-62(2004)). It is speculated that ATP synthase β subunit is an important tissue-specific target in the effector phase of an apoptosis pathway.

The present invention also provides for a pharmaceutical composition comprising an inhibitor of ATP synthase activity. The pharmaceutical composition can further comprise a pharmaceutically acceptable carrier.

Though the inhibitors of the present invention are primarily concerned with the treatment of human subjects, they may also be employed for the treatment of other mammalian subjects such as dogs and cats for veterinary purposes.

For the purpose of treatment of disease, the appropriate dosage of the above inhibitors will depend on the severity and course of disease, the patient's clinical history and response, the toxicity of the inhibitors, and the discretion of the attending physician. The inhibitors are suitably administered to the patient at one time or over a series of treatments. The initial candidate dosage may be administered to a patient. The proper dosage and treatment regime can be established by monitoring the progress of therapy using conventional techniques known to the people skilled of the art.

The following examples are offered by way of illustration and not by way of limitation. The disclosure of all citations in the specification is expressly incorporated herein by reference.

### EXAMPLE

### Example 1

This example describes the identification of breast cancer-related proteins.

In the present invention, molecules critical to the treatment of breast cancer were sought by initially detecting differential expression of proteins in normal cells and the breast tumor cell lines.

a. Breast Cancer Specimens and Protein Extraction

Tissue samples were obtained from breast carcinoma patients at different stages that had undergone surgical resection at National Taiwan University Hospital, Taipei, Taiwan. The breast cancer tissue of the patients and the adjacent normal tissue were collected. The frozen tissue was lyophilized and grinded into powder with liquid N₂ and stored at −80° C. until use. The powdered tissue was dissolved in 1 mL lysis solution containing 7 M urea (Boehringer, Mannheim, Germany), 2 M thiourea, 4% CHAPS (J. T. Baker, Phillipsburg, N.J., USA) and 0.002% bromophenol blue (Amersco, Ohio, USA). The mixture was discontinuously manual sonicated for 5 minutes on ice. The extracted protein concentration was measured by protein assay kit (Bio-Rad, Hercules, Calif., USA).

b. Two Dimensional Gel Electrophoresis (2DE)

In one embodiment, 2DE was performed using Ettan IPGphor II (Amersham Pharmacia Biotech, Uppsala, Sweden). 500 μg of total proteins were mixed with rehydration buffer containing 7 M urea (Boehringer), 2 M thiourea, 4% CHAPS (J. T. Baker), 65 mM DTE, 0.5% pH 3-10 NL IPG Buffer and 0.002% bromophenol blue (Amersco) to a total volume of 350 uL. The mixtures were loaded onto an 18 cm pH 3-11 NL gradient Immobiline DryStrip (Amersham Pharmacia Biotech). IEF parameters for separation were 50 μA/strip at 20 C with a rehydration step for 12 h. IEF was carried out using the following conditions: (1) 100 V for 1 h; (2) 250 V for 1 h; (3) 500 V for 1 h; (4) 1,000 V for 1 h; (5) 4,000 V for 1 h; (6) and 8,000 V for 85,000 Vh. After reduction with 65 mM DTE and alkylation with 55 mM iodoacetamide, the second-dimensional separation was performed on a linear gradient 10-18% polyacrylamide gel. The protein gel were fixed in 10% methanol/7% acetic acid and stained using the SYPRO® Ruby method (Invitrogen Corporation, Carlsbad, Calif.). Gels were then scanned using a Typhoon 9200™ Fluorescence Imager (Amersham Pharmacia Biotech) and analyzed by Image Master™ 2D elite software package (Amersham Pharmacia Biotech) using high image quality TIF format.

c. In-Gel Digestion and Protein Identification

By gel-to-gel comparison, the 2D image of the tumor tissue was set as the reference gel image. After matching the normal tissue gel image to the reference image, only the protein spots displayed on the tumor tissue gel were excised. The gel pieces were washed with 1:1 (v/v) solution containing 50 mM ammonium bicarbonate and ACN. After treatment with Na₂CO₃, proteins were digested for 16 h at 37° C. with sequence-grade trypsin (Promega Corporation, Wis., USA). The resulting peptides were extracted from the gel with 1% TFA in 50% ACN. The combined extracts were evaporated to dryness and the protein fragments were dissolved in 0.1% TFA in 2% ACN and directly spotted onto the sample plate of a MALDI-TOF mass spectrometer.

MALDI-TOF MS or MS/MS were performed on a dedicated Q-Tof Ultima MALDI instrument (Micromass, Manchester, UK) with fully automated data directed acquisition using predefined probe motion pattern and peak intensity threshold for switching over from MS survey scan to MS/MS, and from one MS/MS to another. All individual MS/MS data thus generated from a particular sample well were then output as a single MASCOT-searchable peak list file. Within each sample well, parent ions that met the predefined criteria (any peak within the m/z 800-3000 range with intensity above 10 count±include/exclude list) were selected for CID MS/MS using argon as the collision gas and a mass dependent ±5 V rolling collision energy until the end of the probe pattern was reached. Subsequently, protein identification was determined by searching in the SWISS-PROT version 51.7 database using the MASCOT search engine. All the searching parameters were set up as follows: peptide mass tolerance was ±50 ppm; fragment mass tolerance was ±0.25 Da; only tryptic peptides up to one missed cleavage site was allowed; modifications were carbamidomethylation (C) and oxidation of methionine. For positive identification, the score of the result of [−10 Log(P)] had to be over the significance threshold level (P<0.05).

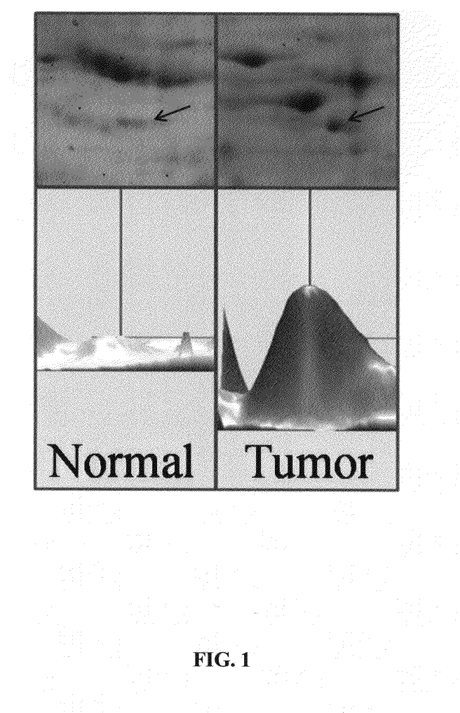

Proteins extracted from the same patient tissue section were separated using 2DE. Approximately 1,000 protein spots were visualized by staining with Sypro® Ruby. Differentially expressed protein spots were excised, in-gel digested and analyzed using MALDI Q/TOF. After database searching, 38 distinct proteins were identified. From the results, proteins involved in anti-apoptosis, cell motility, cell proliferation, glycolysis, protein folding, signal transduction and other processes related to tumorgenesis were found to be overexpressed in cancer tissues. Among them, significant upregulation of ATP synthase β subunit was observed in breast cancer tissue (denoted by an arrow in FIG. 1).

### Example 2

This example describes the treatment of breast cancer by using the inhibitors of ATP synthase.

a. Cell Culture

Human breast cancer cell lines, MCF-7, MDA-MB231, T47D and human normal breast cell line MCF-10A, were obtained from the American Type Culture Collection (Manassas, Va.). Human breast cancer cell lines were maintained in DMEM at 37° C. with 5% CO₂ (Gibco, Carlsbad, Calif., USA), 5% fetal bovine serum (Gibco), 50 units/mL penicillin, and 50 μg/mL streptomycin (Gibco). MCF-10A cells were culture in DMEM (Gibco), 0.01 mg/mL Insulin, 5 μg/mL Hydrocortisone.

b. Flow Cytometry

Cells were harvested washed with PBS and incubated with monoclonal anti-β-subunit antibody (Abcam, Cambridge, UK) (1:500) for 30 min. Cells were then washed twice with PBS and incubated with a secondary goat anti-mouse antibody-FITC (Chemicon Inc, MA, USA) in the dark for 30 min. All antibody incubations were carried out at 4° C. in PBS with 1% BSA. The mean fluorescent intensity of FITC in 10,000 cells was quantified by FACSCalibur flow cytometer (Becton Dickinson, Franklin Lakes, N.J., USA).

c. Confocal Microscopy

Cells were plated at 150,000 cells/mL on glass coverslips and allowed to adhere overnight. Cells were fixed in 4% paraformaldehyde solution at room temperature. A control slide was permeabilized in 1% Triton X-100 for 30 min at room temperature after fixation. All coverslips were incubated in 0.1% BSA in Dulbecco's PBS overnight at 4° C. and washed before incubation with monoclonal anti-β-subunit antibody (Abcam) (1:250) for 1 hr. All cells were washed three times and incubated for 30 min in the dark at room temperature with a secondary goat anti-mouse antibody-PE (1:100). After the final washes, cells were visualized by using a Zeiss LSM-510 (Switzerland) confocal microscope.

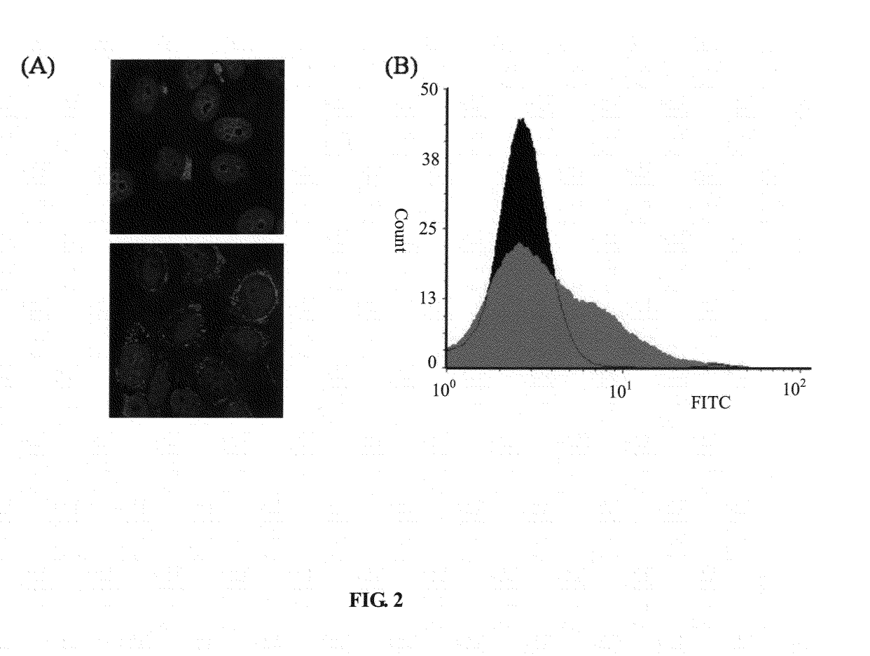

To investigate the localization of the ATP synthase, breast tumor cell line MCF-7 was analyzed by using confocal microscopy with a mAb specific for the β-subunit of ATP synthase. Each cell displayed one or more irregular clusters of punctuated structures, suggesting an organized distribution of the β-subunits of ATP synthase on the cell surface. As shown in FIG. 2A, permeabilized cells produced a considerably different pattern characteristic of mitochondrial staining of ATP synthase. Similar analysis of the breast cell line MCF-7 by flow cytometry also showed that these cells have ATP synthase β subunit on their cell surface (FIG. 2B).

d. Homology Modeling

The three-dimensional model of ATP synthase was generated with the MODELLER program encoded in InsightII (Accelrys, Inc, California, USA) by using bovine ATP synthase (Protein Data Bank code 1H8E) as the template structure. MODELLER uses a spatial restraint method to build up a 3-D image of the protein structure. MODELLER is capable of generating a reliable predicted structure using probability density functions derived from homologous structures and general features of known proteins. For ATP synthase protein alignment, MODELLER yielded only one model with high similarity to the bovine template. The coordinates of the high resolution structure of bovine ATP synthase were used to model the main chain conformation of human ATP synthase. The structure with the lowest violation score and lowest energy score was chosen as the candidate.

e. Docking Simulation

In order to further explore possible interaction of drug candidates with ATP synthase, a docking experiment was performed using the receptor molecular model and docking protocol. This protocol consists in the flexible fitting of ligand (aurovertin B, resveratrol and picetanol) within a rigid receptor (ATP synthase, homologues to PDB entry 1H8E) using the shape-based docking algorithm LigandFit. The obtained poses were subsequently scored using the LigScore scoring function. The best pose was then energy minimized with CHARMm allowing full flexibility for the ligand and only side-chain flexibility for the receptor. All calculations were carried out in the Discovery Studio 1.2 environment (Accelrys, Inc).

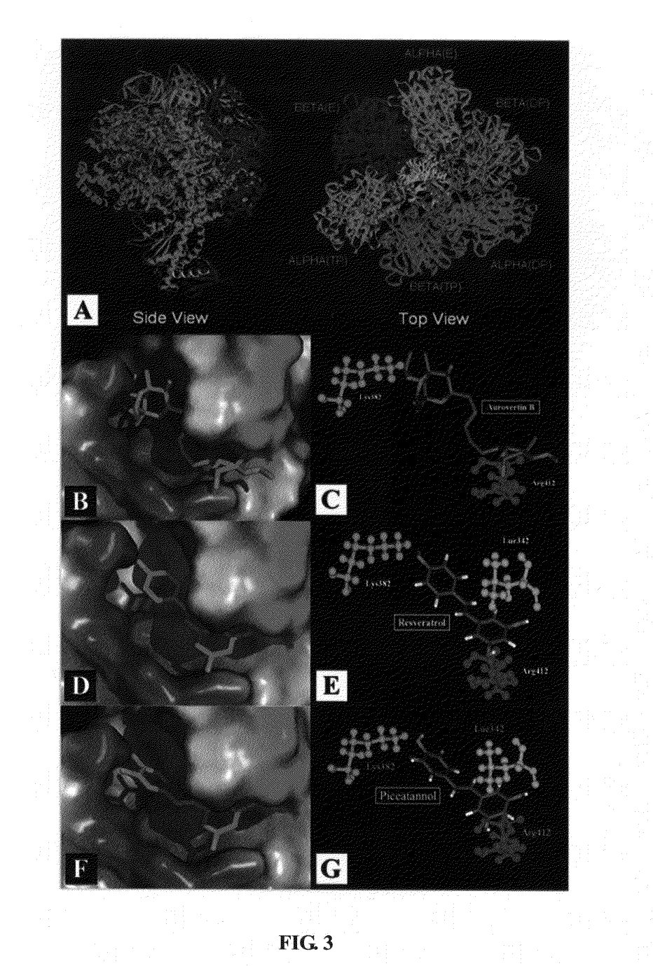

Homology modeling of bovine ATP synthase combined docking stimulations using three inhibitors further confirmed the correlation between the ATP synthase and breast cancer. An optimal sequence alignment is essential to the success of 120 homology modeling. The sequence identity between human and bovine ATP synthase is 99% (data not shown), making this sequence alignment relatively straightforward. After energy minimization, the modeled structure shown in FIG. 3A was exhibited as a reliable protein structure prediction. Using this structure, three ligands were used for docking simulations: aurovertin B (FIG. 3B and FIG. 3C), resveratrol (FIG. 3D and FIG. 3E) and piceatannol (FIG. 3F and FIG. 3G), respectively. These three compounds demonstrated high affinity and formed hydrogen bonds with the Lys382, Arg412 and Lue342 residues of the ATP synthase β subunit. The docking results showed a firm binding affinity of aurovertin B to ATP synthase (aurovertin B=79.803, Resveratrol=35.889, Piceatannol=44.364). Aurovertin B can dock into ATP synthase β subunit. Arg-412 makes an important hydrogen bonding interaction with the carbonyl group on the substituted pyrone ring of aurovertin B and Tyr-458 forms a crucial staggered stacking interaction with the pyrone ring.

f. MTT Assay

Cytotoxic effects on the growth and viability of cells were determined using MTT (3(4,5-dimethylthiazol-2-yl)-2,5-diphenyltetrazolium bromide, a tetrazole) assay. 10⁴ cells/mL were plated in 96-well plates and allowed to attach for 24 hours. Cells were treated with 0.1 to 10 μm of aurovertin B, resveratrol and piceatannol for 48 hours. At the end of the incubation period, MTT reagent (10 μL) was added to each 100 μL of culture. After incubation for 4 hr at 37° C., the formed water insoluble formazan dye was solubilized by adding 100 μL of DMSO to the culture wells. The plates were further incubated for 15 min at room temperature, and optical density (OD) of the wells was determined using ELISA microplate reader at a test wavelength of 570 nm. All experiments were performed in triplicates.

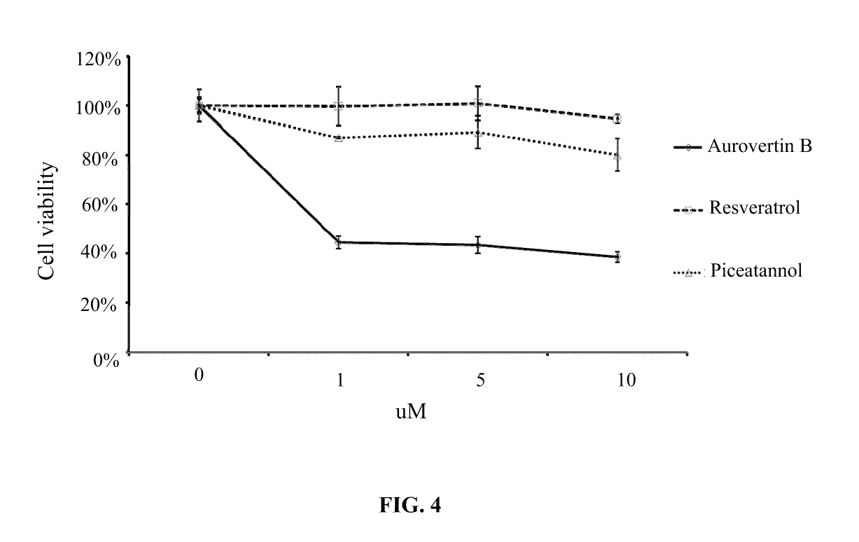

As shown in FIG. 4, a cell proliferation assay was in the presence of F₁-targeting H⁺-ATP synthase inhibitors. MTT assay was performed to characterize the specificity of ATP synthase inhibitors on tumor cells. MCF-7 cells were treated with three chemicals, seprarately, for 48 hours. Aurovertin B caused significant apotosis in MCF-7. On the other hand, cells treated with the other two ATP synthase inhibitors (resveratol and piceatannol) had 80% and higher cell viability rate at the same concentrations and the same time points compared with aurovertin B. Aurovertin B inhibited the growth of MCF-7 cells in a dose-dependent manner. Aurovertin B was the most potent of the inhibitors with an IC₅₀ value at 0.1 μM. Resveratrol and piceatannol were slightly less effective than aurovertin B. However, aurovertin B can't cause apotosis in HeLa cervical cancer cells, as described in Konstantin G. Lyamzaev, “Inhibition of mitochondrial bioenergetics: the effects on structure of mitochondria in the cell and on apoptosis,” (Acta Biochimica Polonica, Vol. 51: 553-562 (2004)).

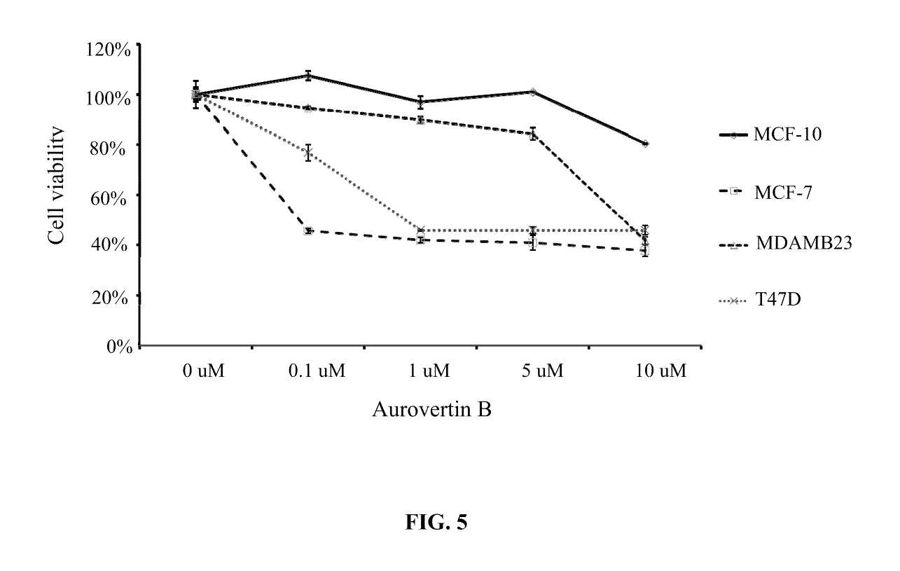

The selective cytotoxicity of aurovertin B to cancer cells can be demonstrated in breast cancer cell lines T47D, MDAMB231 and MCF-7, as well as normal breast cell line MCF-10A by MTT assay to measure the cell viability, as shown in FIG. 5. The IC₅₀ values of T47D, MDAMB231 and MCF-7 were 0.89 μM, 5.52 μM and 0.09 μM, respectively, after aurovertin B treatment for 48 hours. Aurovertin B also caused significant apotosis in breast cancer cell lines. However, normal breast cell line MCF-10A cells treated with aurovertin B had 80% or higher cell viability rate at the same time points, meaning it exerts little toxicity to normal cells. The results showed no apparent cytotoxicity to normal human breast cell line MCF-10A under these concentrations.

g. Cell Cycle Analysis

For the determination of cell cycle phase distribution, 3×10⁵ MCF-7 cells were first exposed to various concentrations of aurovertin B in DMEM with 10% FBS for 48 hr by flow cytometric analysis. The cells were trypsinized, collected and fixed in 70% cold ethanol (−20° C.) overnight. After washing twice with PBS, cells were resuspended in PBS. RNase A (1 mg/mL) and PI (10 μg/mL) were added to the fixed cells for 30 min. The DNA content of cells was then analyzed with a FACSCanto instrument (Becton Dickinson, San Jose, Calif., USA). The percentage of cells in different phases of the cell cycle was calculated by MultiCycle (DeNovo software, Ontario, Canada).

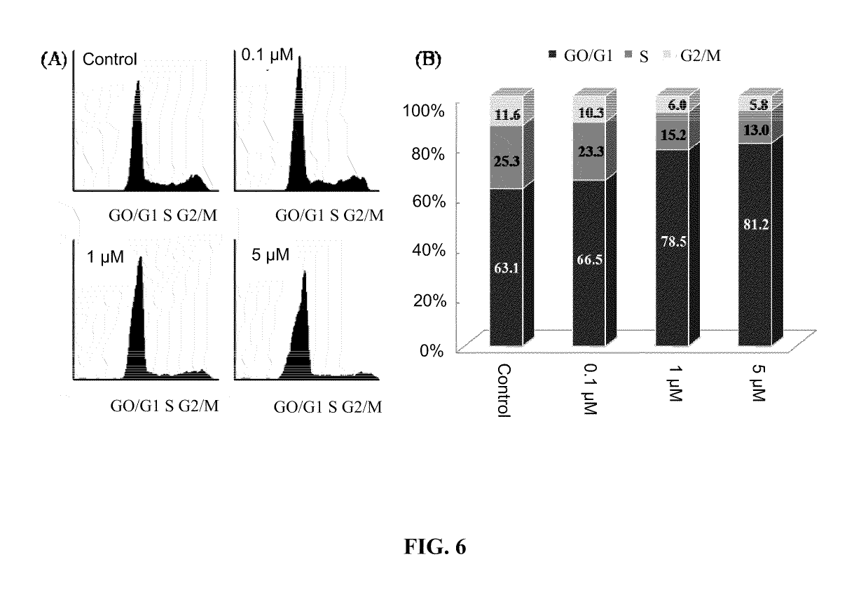

As shown in FIG. 6A, aurovertin B inhibits MCF-7 tumor cell growth by arresting cell cycle at the G0/G1 phase. In order to investigate the effect of aurovertin B on cell-cycle progression of MCF-7 cells, their DNA content was analyzed by flow cytometry, and the derived data was used to investigate the phase distribution of the cell cycle. Cell cycle analysis of MCF-7 cells treated with 0.1 μM, 1 μM and 5 μM aurovertin B for 48 hours was performed by flow cytometry. As shown in FIG. 6B, the investigated MCF-7 cells revealed a cell-cycle distribution typical of that in rapidly proliferating cells, with 63.1% of cells featuring a 2n DNA content, corresponding to the G0/G1-phase, 11.6% of cells exhibiting a 4n DNA content (G2/M) and 25.3% with a DNA content between 2n and 4n, corresponding to the S-phase. MCF-7 cells cultured in the medium without aurovertin B for 48 hours served as the control. In aurovertin B treated cells, percentage of G0/G1 phase was increased to 63.1% to 66.5%, 78.5%, 81.2% under the respective concentrations in a dose-dependent manner.

h. Annexin V-FITC/PI Analysis

Flow cytometric analysis was performed to identify and quantify the apoptotic cells by using Annexin V-FITC/PI (propidium iodide) apoptosis detection kit (Santa Cruz Biotechnology, Santa Cruz, Calif., USA). Cells were treated with 0.1 to 10 μm of aurovertin B for 48 h. After incubation, floating and adherent cells were harvested. Cells were washed carefully with cold PBS at 4° C. Cell pellets were resuspended in 100 l of binding buffer. Annexin-V FITC (0.2 μg/100 μL) and PI (10 μg/mL) was added to the cells and left for incubation in the dark for 15 min at room temperature. All data were acquired by flow cytometry using a FACSCanto cytometer (Becton Dickinson, San Jose, Calif., USA). The flow cytometric analysis was performed using FCS Express 3.0 (DeNovo software).

i. DAPI Staining

After treatment for 48 h with DMSO (control) or aurovertin B (at their respective IC50 concentrations), cells were fixed in 4% paraformaldehyde for 15 min and stained with DAPI (2 μg/mL in PBS) for 15 min at 37° C. to detect apoptotic bodies (Sigma, St. Louis, Mo., USA). Results were determined by visual observation of nuclear morphology via fluorescence microscopy.

j. Statistical Analysis

The results are presented as means±SD of at least three independent experiments.

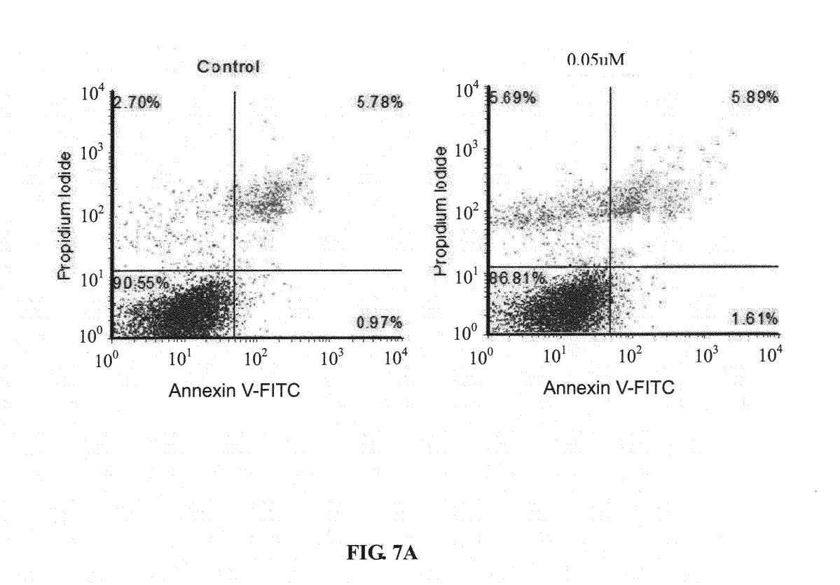

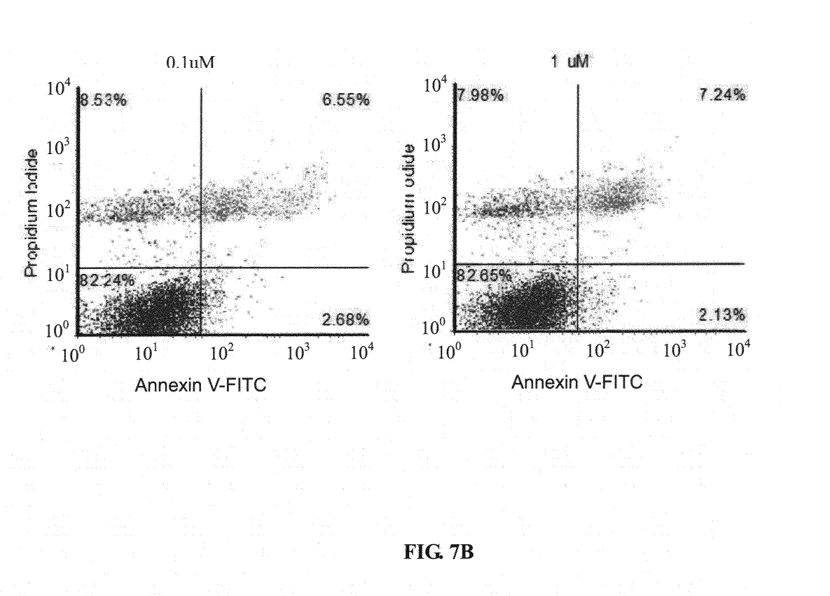

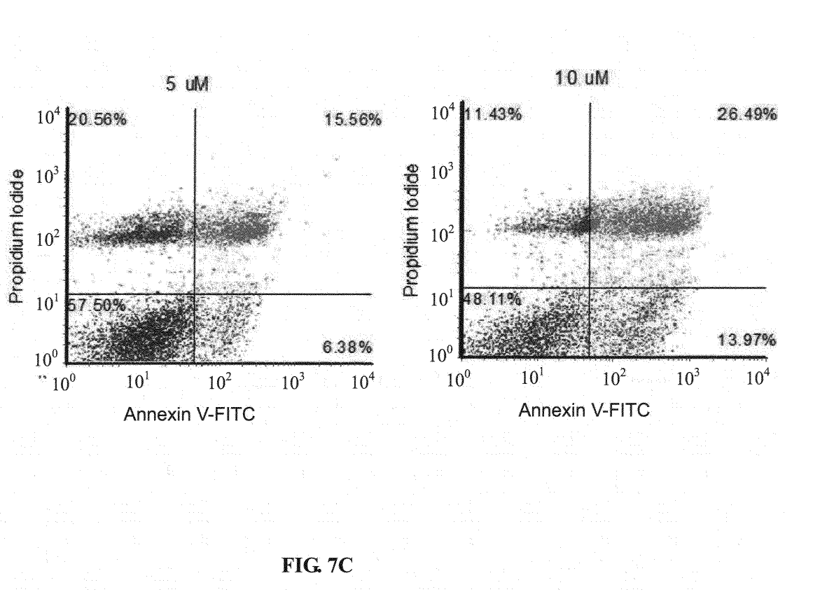

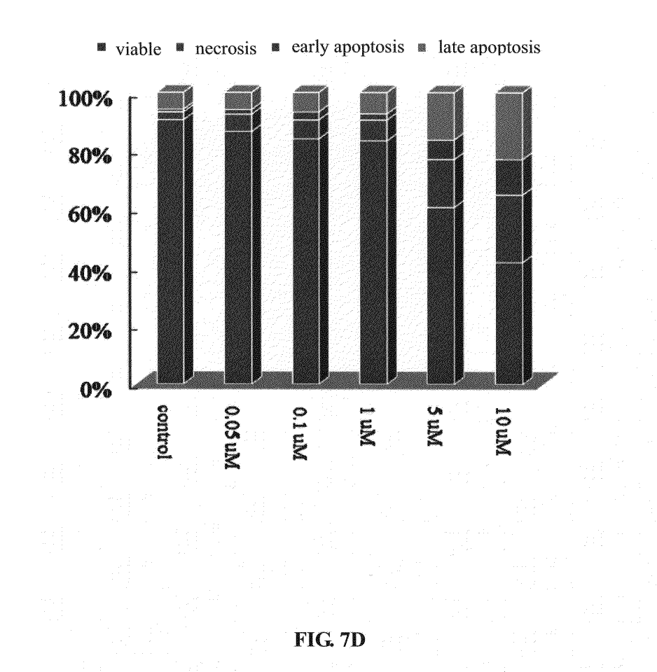

As shown in FIG. 7a, aurovertin B induces apoptosis in human MCF-7 cells. To clarify whether the induced decrease in viability and growth rate was attributable to apoptosis, pattern characteristics of apoptosis were investigated by FITC-annexin V and PI staining and DAPI staining. FITC-annexin V and PI staining assay divides apoptotic cells into two stages: early (Annexin V⁺/PI⁻) and late apoptotic/necrotic (Annexin V⁺/PI⁺). Cell cycle analysis of MCF-7 cells treated with 0.1 μM, 1 μM and 5 μM aurovertin B for 48 hours was performed by flow cytometry. The treatment of cells with aurovertin B (0.05-10 μM) resulted in a dose-dependent increase in both early and late apoptotic/necrotic cells. As shown in FIG. 7b, the percentage of cells in the four quadrants was calculated. MCF-7 cells cultured in the medium without aurovertin B for 48 hours served as the control.

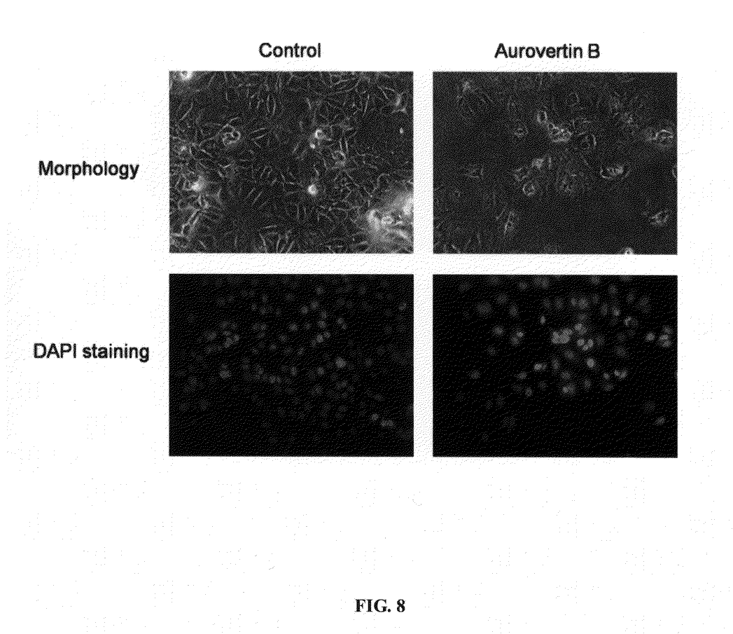

As shown in FIG. 8, aurovertin B induces cell death in human MCF-7 cells. The morphological changes of the nuclei in cells treated with aurovertin B were examined by nuclear staining, typical apoptotic nuclear changes, such as condensation and shrinkage of nuclei, were observed in MCF-7 cells exposed to aurovertin B.

### Example 3

This example describes the identification of lung cancer-related proteins.

In the present invention, molecules critical to the treatment of lung cancer were sought by initially detecting differential expression of proteins in normal cells and the lung tumor cell lines.

Tissue samples were obtained from non-small cell lung carcinoma patients that had undergone surgical resection at Taichung Veterans General Hospital, Taichung, Taiwan. The lung cancer tissue of the patients and the adjacent normal tissue were collected. The processes of proteins purification and analysis were as description of Example 1, and finally the significant upregulation of ATP synthase β subunit was observed in lung cancer tissue.

### Example 4

This example describes the treatment of lung cancer by using the inhibitors of ATP synthase.

Apart from the traditional role in ATP production, ATP synthase may play critical roles in tumor cell metastases. Human lung adenocarcinoma cell lines CL1-1 and CL1-5 with different metastasis were obtained from the American Type Culture Collection (Manassas, Va.), and human normal lung cell line IMR-90 was obtained from Institute of Biomedical Sciences, Academia Sinica. The CL1-5 cell line is highly metastatic lung cancer cells, but the CL1-1 cell line is low metastatic lung cancer cells.

Human lung adenocarcinoma cell lines were maintained in DMEM at 37° C. with 5% CO₂ (Gibco, Carlsbad, Calif., USA), 5% fetal bovine serum (Gibco), 50 units/mL penicillin, and 50 μg/mL streptomycin (Gibco). IMR-90 cells were culture in DMEM (Gibco), 0.01 mg/mL Insulin, 5 μg/mL Hydrocortisone.

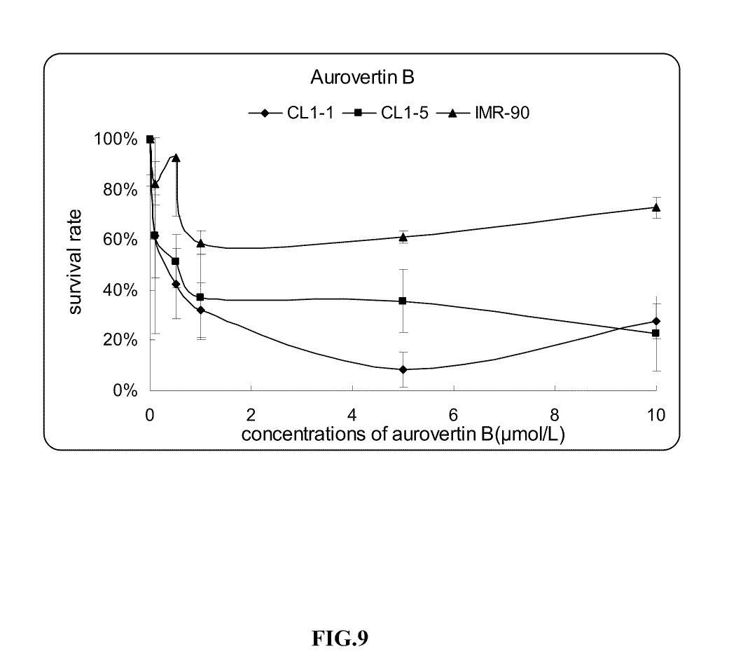

As shown in FIG. 9 to FIG. 11, cell proliferation assays were in the presence of three F₁-targeting H-ATP synthase inhibitors: aurovertin B, resveratol, and piceatannol. By counting viable cell number using trypan blue, the inhibitors were demonstrated with anti-cancer effects on cell survival rates using cell lines of CL1-1, CL1-5, and IMR-90. As shown in FIG. 9, CL1-1 cells treated with aurovertin B had an IC₅₀ value at 0.1 μM to 0.5 μM and CL1-5 cells treated with aurovertin B had an IC₅₀ value at about 0.5 μM. Thus, aurovertin B caused significant apotosis in CL1-1 and CL1-5 cells.

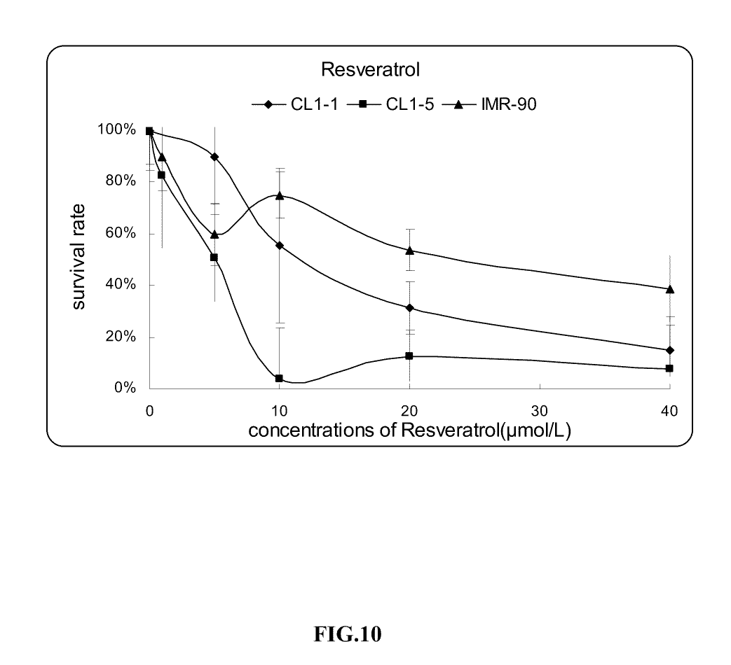

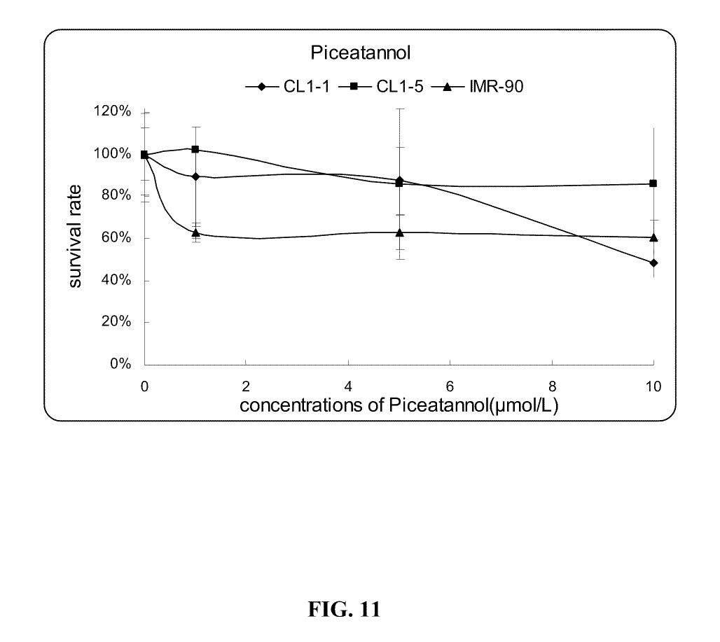

As shown in FIG. 10, CL1-1 cells treated with resveratol had an IC₅₀ value at 10 μM and CL1-5 cells treated with resveratol had an IC₅₀ value at about 5 μM. On the other hand, CL1-1 cells and CL1-5 cells treated with piceatannol showed no significant apotosis as shown in FIG. 11. Aurovertin B inhibited the growth of CL1-1 and CL1-5 cells in a dose-dependent manner. Aurovertin B was the most potent of the inhibitors with an IC₅₀ value at 0.5 μM. Resveratrol was slightly less effective than aurovertin B, and piceatannol has no effect in lung adenocarcinoma cells (CL1-1 and CL1-5).

These experimental results suggest that aurovertin B can cause more cell death in low metastatic CL1-1 lung cancer cells than in highly metastatic CL1-5 lung cancer cells. However, highly metastatic CL1-5 lung cancer cells treated with resveratrol demonstrated a much higher percentage of cell death than low metastatic CL1-1 lung cancer cells. It is speculated that aurovertin B and resveratrol cause different cytolytic pathways to induce the cell death. In addition, resveratrol may inhibit tumor cell metastases.

The above experiments demonstrate that ATP synthase expression is closely related to certain cancer cells. It is localized on the plasma membrane of breast and lung cancer cells, and may play a key role in the biological activities of breast and lung cancer cells. Blocking the activity of ATP synthase by aurovertin B, binds with ATP synthase F₁ subunit, inhibits the breast cancer cell growth and proliferation and induces cell death in breast and lung cancer cells, suggesting the possibility of clinical application of ATP synthase inhibitors as anti-tumor agents for treating breast cancer and lung cancer.

Although the invention has been described with reference to the presently preferred embodiments, it should be understood that various modifications can be made without departing from the spirit of the invention.

What is claimed is:

1. A method of treating cancer comprising administering to a subject an inhibitor of ATP synthase activity in a pharmaceutically effective amount, wherein the inhibitor is aurovertin B, wherein the cancer is breast cancer or lung cancer; and wherein the inhibitor down-regulates biological activities of ATP synthase and induces apoptosis in the cancer.

2. (canceled)

3. The method of claim 1, wherein the cancer is located in a human subject.

4. The method of claim 1, wherein the cancer is resistant to anticancer agent and/or radiation therapy.

5. (canceled)

6. (canceled)

7. (canceled)

8. A method for inducing apoptosis in a cancer cell comprising contacting the tumor cell with an inhibitor of ATP synthase activity, wherein the inhibitor is aurovertin B, wherein the cancer is breast cancer or lung cancer, and wherein the inhibitor down-regulates biological activities of ATP synthase.

9. (canceled)

10. The method of claim 8, wherein the cancer cell is located in a human subject.

11. The method of claim 10, wherein the cancer cell is reduced in the human subject.

12. The method of claim 8, wherein the cancer cell is resistant to anticancer agent and/or radiation therapy.

13. The method of claim 8, wherein the inhibitor directly interacts with ATP synthase F1 subunit.

14. (canceled)

15. (canceled)

16. (canceled)

17. A pharmaceutical composition for inducing apoptosis in breast cancer or lung cancer, comprising an inhibitor of ATP synthase.

18. The pharmaceutical composition of claim 17, wherein the inhibitor is aurovertin B.
